(19) United States
(12) Patent Application Publication
(10) Pub. No.: US 2008/0202214 A1
(43) Pub. Date: Aug. 28, 2008

(54) CRYSTALLIZATION POINT AUTOMATED TEST APPARATUS

(75) Inventors: Kenneth Slater, Houston, TX (US); Marian Baranowski, Houston, TX (US); Arkadiy Belkin, Houston, TX (US)

Correspondence Address: LEGAL DEPT., IP GROUP;M-I L.L.C., 5950 NORTH COURSE DRIVE, HOUSTON, TX, 77072, US

(73) Assignee: M-I L.L.C., Houston, TX (US)

(21) Appl. No.: 11/678,037

(22) Filed: Feb. 22, 2007

(51) Int. Cl. G01N 33/00; B01L 11/00

(52) U.S. Cl. 73/53.01

(57) ABSTRACT

An apparatus for optically detecting the degree of crystallization in a fluid sample while simultaneously measuring the temperature of the fluid sample during cooling or heating of the sample is disclosed. The apparatus includes: a test vial for containing a fluid sample in an interior of the vial, wherein the test vial is light permeable; a cooling source for cooling the fluid sample; a temperature sensor positioned in the interior of the test vial for measuring the temperature of the fluid sample; an external light source configured to direct light into the test vial; and an external light detector configured to measure the amount of light that traverses both the test vial and the fluid sample.

### BACKGROUND

Brine fluids are commonly used as completion, workover, and drilling fluids during subterranean well operations. Brines are aqueous solutions of one or more salts. The salts are typically chlorides, bromides, or formats such as sodium chloride, calcium chloride, calcium bromide, potassium chloride, potassium formate, and sodium formate to name a few. Brines are formulated with a salt density typically in a range from about 8 to about 20 lb/gal depending on the particular use and specific conditions. Brines are commonly used for pressure control because of their non-damaging character as solids free solutions that contain no particles that may damage or plug a producing formation. As such, the density and crystallization temperature of a brine are important specified parameters in normal industry practice.

It is well known that the use of brines in low temperature conditions presents a problem of brine crystallization. At temperatures at or below the crystallization temperature of the brine, the precipitation of crystallizing solids (e.g., salts) can change the density of the brine fluid and deteriorate the ability of the fluid to maintain pressure control. Further crystallization may also lead to crystallized solids plugging the filters and lines in the subterranean well. Thus the crystallization temperature of a brine fluid is an essential parameter to know for low temperature applications in cold climates.

The crystallization temperature of a brine is commonly measured in accordance to a standardized test method described in ANSI/API Recommended Practice 13J, entitled “Testing of Heavy Brines”, 4^{th} Ed. (May 2006). To characterize the crystallization profile of the brine, as described in API Recommended Practice 13J, an apparatus is used to alternately cool and heat a sample of brine fluid for measuring three different crystallization temperatures. During testing, the sample is slowly and continuously cooled until a temperature is reached at which visible crystals start to form in the sample and the temperature is recorded as the First Crystal to Appear (FCTA) temperature. During cooling, the FCTA temperature corresponds to a minimum inflection point in a plot of temperature versus time, the minimum inflection point being generally the result of a super-cooling effect. Upon reaching the FCTA temperature, the cooling temperature is held constant while the exothermic brine crystallization process proceeds. Heat is released during the brine crystallization process and the maximum temperature, or maximum inflection point, reached immediately following the FCTA temperature is recorded as the True Crystallization Temperature (TCT). The TCT corresponds to the actual true crystallization temperature of the brine. After obtaining the TCT, cooling is discontinued and the brine is allowed to warm, or is heated, to dissolve the crystals. The temperature at which the last crystal is observed to disappear is recorded as the Last Crystal to Dissolve (LCTD) temperature. The LCTD temperature also corresponds to a minimum inflection point due to an increase in the heating rate of the brine just after the crystals have completely dissolved.

According to ANSI/API Recommended Practice 13J, it is recommended that the cooling/heating testing described above is repeated at least three times for a given brine sample and the average measurements are reported as the FCTA, TCT, and LCTD temperatures for the brine. The accuracy of the FCTA, TCT, and LCTD measurements is, in part, affected by the rate of cooling, rate of heating, and visual observation of crystallization. Visual inspection of the brine sample during testing enhances accuracy because one or more of the crystallization event inflection points on a temperature versus time plot is often subtle and difficult to identify.

The apparatus of the present invention provides an automated crystallization point test apparatus as an alternative apparatus to those described in the prior art. In particular, one such apparatus described in the prior art uses a fiberoptic probe for optically detecting crystallization. In the preferred configuration, a fiberoptic probe with a closely-spaced mirror is immersed in a sample solution to detect crystals across a small portion of the sample solution. Optically examining only a small volume of the sample is undesirable in that it limits the accuracy particularly with respect to detecting the first crystal to appear and the last crystal to dissolve during FCTA and LCTD measurements. Another disadvantage of utilizing an immersed fiberoptic probe is the potential for fouling the tip of the probe and/or mirror submerged in the sample solution, which may adversely affect accuracy and reproducibility of measurements. Additionally, the presence of the immersed probe undesirably interferes with the circulation of the sample during stirring. In addition to these disadvantages or limitations, is the relatively expensive cost of a fiberoptic probe.

Despite efforts in the prior art, there is a need for an automated apparatus and method that provides highly accurate and reproducible crystallization temperature measurements.

### SUMMARY

The subject matter of the present disclosure is generally directed to an apparatus for measuring the crystallization temperatures of a fluid. The present invention provides an automated apparatus having an optical capability for detecting crystals in the sample solution, thus eliminating the need for a person to visually observe the sample for the presence of crystals when measuring the crystallization events FCTA, TCT, and LCTD of a brine sample. Another advantage of the present invention is that the optical technique employed enhances the accuracy in determining the FCTA and LCTD temperatures due to its high sensitivity in detecting crystals in solution. Furthermore, the optical technique detects crystallization in a sufficient volume of the brine sample which allows for higher measurement accuracy and reproducibility particularly with respect to detecting the first crystal and last crystal to dissolve temperature measurements FCTA and LCTD.

The apparatus of the invention comprises: a test vial for containing a fluid sample in an interior of the vial, wherein the test vial is light permeable; a cooling source for cooling the fluid sample; a temperature sensor positioned in the interior of the test vial for measuring the temperature of the fluid sample; an external light source configured to direct light into the test vial; and an external light detector configured to measure the amount of light that traverses both the test vial and the fluid sample.

These and other features are more fully set forth in the following description of preferred or illustrative embodiments of the disclosed and claimed subject matter.

### BRIEF DESCRIPTION OF THE DRAWINGS

So that the manner in which the above recited features, advantages and objects of the present invention are attained and can be understood in detail, a more particular description of the invention, briefly summarized above, may be had by reference to the embodiments thereof which are illustrated in the appended drawings. It is to be noted, however, that the appended drawings illustrate only typical embodiments of this invention and are therefore not to be considered limiting of its scope, for the invention may admit to other equally effective embodiments.

FIG. 1 illustratively depicts a partial cross-sectional view of one embodiment of the apparatus of the present invention.

FIG. 2 illustratively depicts a partial cross-sectional view of another embodiment of the apparatus of the present invention.

FIG. 3 illustrates typical crystallization profiles of a brine sample using the apparatus of the present invention.

### DETAILED DESCRIPTION

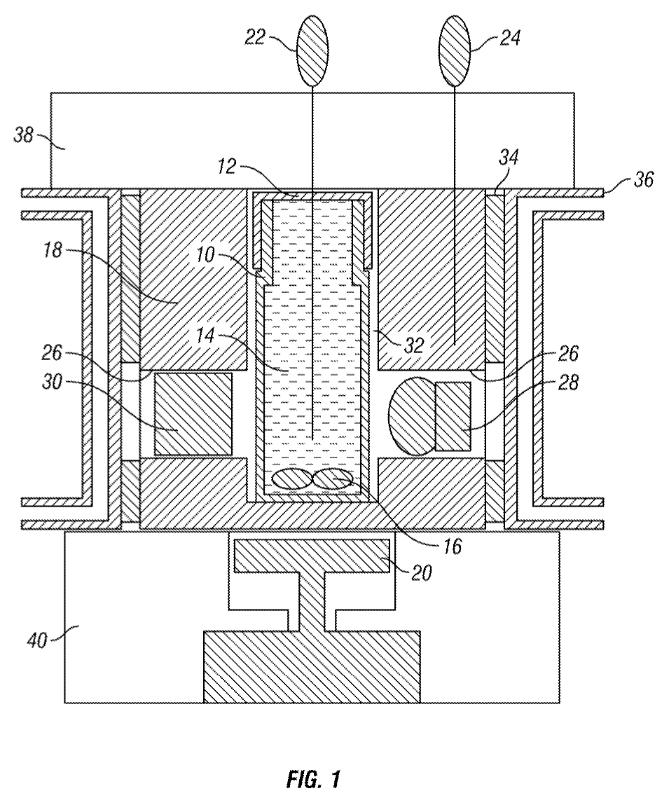

FIG. 1 is a schematic illustration of an embodiment of the apparatus of the present invention equipped with optical instrumentation to carry out a test method for determining the crystallization events of a fluid. The apparatus comprises a test vial 10 and a cap 12 for containing a brine fluid sample 14 and a magnetic stir-pill 16 therein. The body of the test vial 10 should be transparent and optically clear to enable light to pass through the vial 10. Suitable test vial 10 materials include glass and plastic. Preferably, the test vial 10 is a standard clear glass jar or vessel that is commercially widely available. The size of the test vial 10 is selected such that the brine sample 14 fills the vial 10 to a level so as to limit exposure to the atmosphere, for reducing the potential of contamination, and to ensure that the mixing vortex formed during stirring is not in the path of the light that traverses the vial 10. Preferably, the brine sample 14 completely fills, or nearly completely fills, the volume of the test vial 10. The brine sample volume is typically in a range of about 25 ml to about 75 ml, however any quantity may be used. A temperature-controlled thermal block 18 surrounds the vial 10, at least partially, in order to provide sufficient cooling and heating to the brine sample 14 during testing. The thermal block 18 may be made of metal (e.g., aluminum or copper), metal alloys, or any other thermally conductive material. Furthermore, the test vial 10 containing the magnetic stir-pill 16 is positioned above a magnetic stirring plate 20 for stirring the brine sample 14 during testing. However, stirring the brine sample 14 may be accomplished by other stirrers or stirring systems that should be well known to one of skill in the art.

A temperature sensor 22 extends into the brine fluid sample 14 to measure the temperature of the brine sample 14 during testing. Preferably the temperature sensor 22 is a RTD probe, however other temperature sensors, for example, a thermocouple or a thermometer, may be used. The temperature sensor 22 may be mounted to an interior surface of the cap 12, or otherwise attached to the apparatus, such that it extends into the brine fluid sample 14. Another temperature sensor 24 (e.g., a RTD probe) attached to the thermal block 18 is used to measure the temperature of the thermal block 18 to aid in its temperature control.

To optically detect the presence of crystals in the brine sample 14, the temperature-controlled thermal block 18 has a lateral through-hole 26 therein to accommodate optical instrumentation comprising an external light source 28 and an external light detector 30. The light source 28 and light detector 30 are externally positioned outside the test vial 10 and laterally spaced such that the path of a light beam emanating from the light source 28 is directed into the test vial 10 and towards the light detector 30. A suitable light source includes light emitters such as lasers, lamps, LEDs, or any other light emitter that can transmit light across the test vial 10 and into the light detector 30. The light emanating from the light source 28 may be essentially any type of light including visible, polarized, laser, IR, and UV. Likewise, suitable light detectors include a photo-resistor, photo-transistor, photodiode, photovoltaic cell, and other detectors that should be familiar to one of skill in the art.

Furthermore, the optical instrumentation is preferably configured such that the light beam may travel in a single pass from the light source 28, through the vial 10 and a portion of the brine sample 14 therein, and then into the light detector 30. This configuration allows for detection of crystals in the portion of the brine sample through which the light travels as the light traverses the vial 10. Thus, while the brine sample 14 is continuously stirred during testing, the portion of the brine sample that is optically detectable is the constant volume of the brine sample in the path of the light beam. The portion of the brine sample 14 optically monitored should be a sufficient volume for providing high accuracy in optically detecting the presence of crystals in fluids containing only a very dilute concentration of crystals, for example when detecting the FCTA and LCTD temperatures. For typical 25 ml to 75 ml brine samples, the portion of the brine sample 14 optically monitored is preferably equal to a volume of about 5% or more of the total brine sample volume. More preferably, the portion of the brine sample 14 optically monitored is a volume in a range from about 10% to about 30% of the total brine sample volume. Thus, for ensuring high accuracy in detecting the degree of crystallization in dilute crystal solutions, the optical instrumentation should be configured to optically monitor at least about 2 ml of the brine sample 14, and more preferably from about 4 ml to about 10 ml of the brine sample 14. In general, increasing the amount of volume optically monitored increases the accuracy of the optical technique in detecting the first crystal to appear and the last crystal to dissolve temperature measurements FCTA and LCTD.

During testing, the light detector 30 detects the presence of crystals by continually measuring the light transmission across the vial 10 and the portion of the brine sample 14 in the path of the light beam. When there are no crystals in the brine fluid sample 14, the emitted beam travels through the vial 10 and optically-clear brine sample 14 and is received by the light detector 30 (e.g., photo-resistor). During cooling, when crystals form in the brine sample 14, light is blocked by the opaque crystals thus reducing the amount of the light beam that passes completely through the sample 14 and into the light detector 30. The attenuated light detected by the light detector 30 is related to the degree of crystallization in the brine sample 14. This optical technique is highly sensitive to detecting crystals in solution. By detecting crystallization across at least about 2 ml of the brine sample 14, this optical technique provides very accurate and reproducible detection of crystallization events FCTA and LCTD.

Optionally, the test vial 10 may be made of a non-transparent material, for example metal, however the vial 10 needs to contain a transparent region in order to allow the single-pass light beam, emanating from the external light source 28, to traverse the vial 10 and the brine sample 14 therein before reaching the external light detector 30. For example, a suitable test vial 10 made of metal may have two transparent windows laterally positioned in opposing sides of the vial wall such that the directed light beam enters the vial by traversing one of the transparent windows, then travels across the brine sample, and subsequently exits the vial by traversing the other transparent window towards the external light detector 30. Alternatively, the brine sample 14 could fill the thermal block 18 without the use (i.e., enclosure) of a test vial 10. For example, the brine sample 14 may directly fill the central cavity of the thermal block 18 and two transparent windows may be positioned in the lateral through-hole 26 of the thermal block 18, i.e., one window adjacent the light source 28 and the other window adjacent the light detector 30, such that the windows contain the brine sample 14 within the central cavity and prevent the sample 14 from contacting the light source 28 and light detector 30.

It should be noted that the present invention does not limit the position of the light detector 30 to a position within the straight path of the light beam, as depicted in FIG. 1. While the light detector 30, positioned in the straight path of the light beam, measures a decrease in light as crystals form in the brine sample 14, conversely, another light detector (not shown) may be used that is positioned, for example, orthogonal to the straight path of the light beam which measures an increase in light due to the scattering of the light blocked by the opaque crystals. Thus, an increase in light detected by the orthogonally-positioned light detector is related to an increase in crystallization of the brine sample. The thermal block 18 may contain one or more additional holes, for example an additional lateral hole located orthogonal to the lateral through-hole 26, in order to accommodate one or more additional light detectors.

As depicted in FIG. 1, the test vial 10 positioned in close proximity to the surrounding temperature-controlled thermal block 18 preferably creates a gap or an insulating layer of air 32 that surrounds the vertical wall region of the test vial 10. The gap not only provides the space tolerance required to facilitate insertion and removal of the vial 10 from the thermal block 18, but also provides an insulating layer that slows the cooling of the vial 10. At one point during the test when cooling is stopped, the insulating layer of air 32 sufficiently slows the heat transfer from the test vial 10 such that the heat generated during the exothermic crystallization effectuates a rise in temperature for TCT measurement. Without the insulating layer of air 32, or when more area of the test vial 10 is in direct contact with the thermal block 18, the heat generated during crystallization may be drawn away from the test vial 10 too quickly, thus making detection of a rise in temperature more difficult.

Peltier junctions 34 provide the cooling source for cooling the thermal block 18 surrounding the test vial 10. The Peltier junction is a thermoelectric cooling device having a cold ceramic plate on one side and a hot ceramic plate on the other side. While heat is drawn away from the cold plate to the hot plate, the hot plate must be cooled. Accordingly, the cold plate side of each of the Peltier junctions 34 contacts the thermal block 18, for cooling the thermal block 18, and the hot plate side contacts a water jacket 36 to dissipate heat drawn to the hot plate side. The water jacket 36 circulates tap water, or room-temperature water, to sufficiently cool the hot plate sides of the Peltier junctions 34. The temperature control of each of the Peltier junctions 34 is achieved by controlling the efficiency at which heat is removed from the hot plate. The relevant and controllable parameters include the flow rate and temperature of the water circulating through the water jacket 36, as well as regulating the power to the Peltier junctions 34. The water-cooled Peltier junctions 34 supply adequate cooling to the thermal block 18 for crystallization testing while also providing cooling that is both environmentally safe and portable for on-site or field use of the apparatus.

Cooling the brine sample 14 may be accomplished by alternative cooling systems that should be familiar to one of skill in the art. For example, instead of using Peltier junctions 34, a cooling jacket (not shown) that circulates chilled water (or coolant) could be placed in contact with, or in close proximity to, the thermal block 18 for controlling its temperature. In another example, a cooling jacket may also be used without the thermal block 18, wherein the cooling jacket is placed in contact with or close proximity to the test vial 10 for cooling. In another example, a cooling bath (not shown) that circulates chilled water, ice, or coolant around the test vial 10, or the thermal block 18, may also be used for cooling. In still another example, a refrigerator may be used to cool the test vial 10 by placing the apparatus in a temperature-controlled refrigerator. Any cooling system or combination of cooling systems may be used to cool the test vial 10.

The apparatus also optionally comprises an insulated top 38 and an insulated base 40 to enhance temperature control of the thermal block 18. Although the particular configuration of the insulation is not important, providing insulation surrounding any otherwise exposed areas of the thermal block 18 is preferable in order to maintain a uniform temperature throughout the block 18. The insulated top 38 and insulated base 40 may be made of any non-conductive material, such as fiberglass, foam or other polymeric material.

Furthermore, the apparatus is connected to a computer (not shown) to automate the test procedure in a manner consistent with the procedure described in ANSI/API Recommended Practice 13J. During the initial set-up, the computer controller software is initialized by providing the cooling and heating rates, a set-point hold temperature above LCTD, and event triggering levels of the light detector. The computer control is designed to automatically adjust the cooling and heating of the brine sample 14 throughout the test. During the test, the computer monitors the real-time temperature of the brine sample 14, the temperature of the thermal block 18, and the light detector's 30 light attenuation data (i.e., degree of crystallization data). The light attenuation data and the temperature of the brine sample 14 are used to automate the proper control over the cooling and heating rates of the brine sample 14 throughout the test.

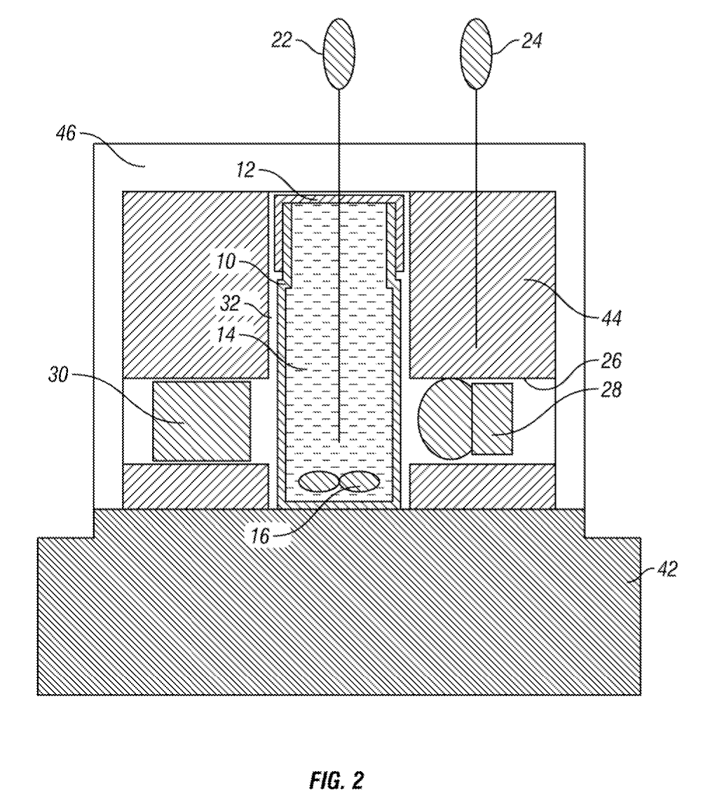

FIG. 2 illustrates another embodiment of the present invention. In FIG. 2 the same reference numerals are used to indicate the same features as those previously described with respect to the apparatus depicted in FIG. 1. In this embodiment, both cooling and stirring of the brine sample 14 is achieved using a cooling magnetic stir-plate 42. Cooling magnetic stir-plates are available commercially from, for example, Digital Stir Kool (model 21485). As depicted, a thermal block 44 contacts the top surface (plate) of the cooling magnetic stir-plate 42 for cooling. Thermal block 44 preferably has an opening in the base of the block 44 for the purpose of allowing the base side of the test vial 10 to also directly contact the cooling magnetic stir-plate 42 to facilitate heat transfer for cooling. Optionally, to further facilitate heat transfer, a heat-transfer grease or oil (not shown) may be used at the interface between the stir-plate 42 and the test vial 10 as well as at the interface between the stir-plate 42 and the thermal block 44. An insulating jacket 46 surrounds the otherwise exposed outer surfaces of the thermal block 44 and cap 12 to enhance temperature uniformity throughout the block 44 and the vial 10. The insulating jacket 46 may be made of any non-conductive material, such as fiberglass, foam or other polymeric material. This embodiment is not limited to any particular design of the thermal block 44. For example, instead of utilizing thermal block 44, thermal block 18 previously described with reference to FIG. 1 containing the test vial 10 may contact the top surface of the cooling magnetic stir-plate 42 for cooling by positioning the base side of thermal block 18 directly onto the stir-plate 42 below. The use of a cooling magnetic stir-plate 42 as the cooling source has the added advantage of contributing to the portability of the apparatus for on-site or field use.

The method of the present invention includes optically detecting the degree of crystallization of a fluid for enhancing the accuracy of the crystallization temperature measurements made in accordance with ANSI/API Recommended Practice 13J, as well as, for automating the test procedure after the initial set-up of the test. During the initial set-up, the brine sample 14 is poured into the test vial 10 containing a small magnetic stir-pill 16 therein. The test vial 10 is covered with cap 12 and thermocouple 22 is positioned into the brine sample 14 to continuously measure the temperature of the brine sample 14. Afterwards, the test vial 10 is positioned into the temperature-controlled thermal block 18, or the thermal block 44, and the light source 28 is activated to form a light beam that travels in a single pass through the test vial 10, and a portion of the brine sample therein, and then into the light detector 30. The computer control software is initialized by entering the desired cooling and heating rates, the set-point hold temperature above LCTD, and the light detector's crystallization event triggering levels. After the initial set-up, the remainder of the test procedure is fully automated.

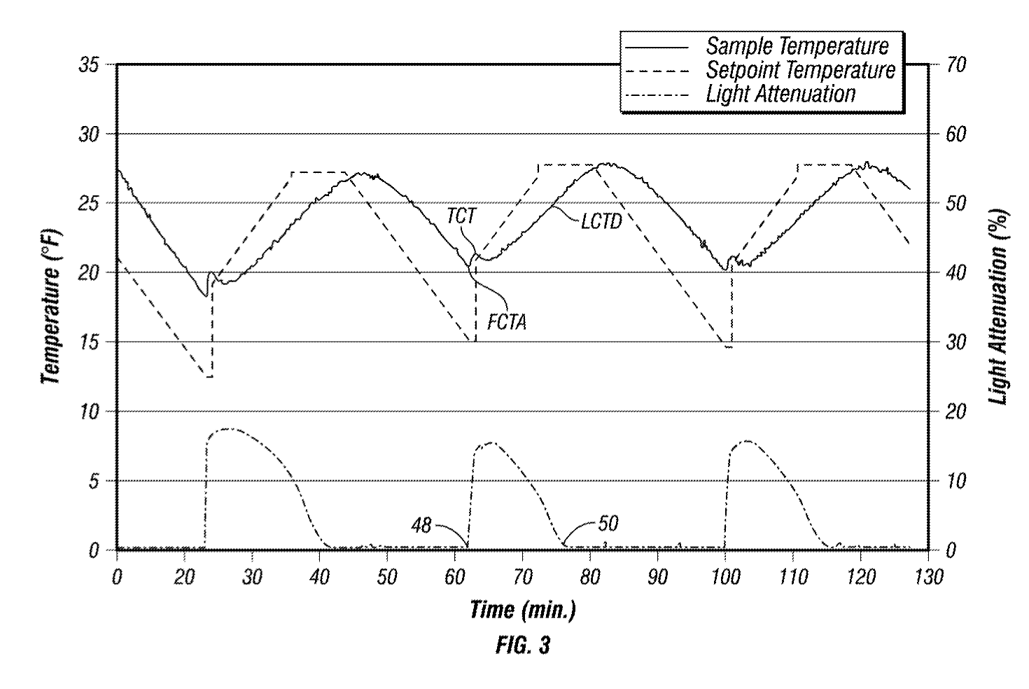

During the automated cooling/heating test, the crystallization profile of the brine sample 14 is monitored by both temperature measurement and attenuated light measurement. FIG. 3 shows the crystallization profiles of three consecutive cooling/heating tests performed with the apparatus of the present invention. Upon starting the test, the brine sample 14 is cooled at the set cooling rate until the onset of crystallization is detected by an attenuation in the light 48 as measured by the light detector. The minimum temperature reached is recorded as the crystallization event FCTA. At this point, cooling is stopped and the temperature held constant while the exothermic crystallization event causes the brine sample 14 temperature to rise. The maximum temperature reached is recorded as the TCT. Subsequently, the temperature is allowed to fall to an intermediate temperature equal to about one-half the difference between TCT and FCTA, and then the brine sample 14 is heated at the set heating rate. Once the LCTD is detected by the light detector as brine sample clarity, i.e., corresponding to a full strength light beam 50, the LCTD temperature is recorded, and the brine sample is heated to the set-point hold temperature above LCTD. Upon inspection of FIG. 3, it should be apparent that the FCTA and the LCTD measurements are particularly difficult to accurately determine without the assistance of the optical technique of the present invention. The automated apparatus of the present invention may be used to provide highly accurate and reproducible FCTA, TCT, and LCTD temperature measurements.

While the foregoing is directed to embodiments of the present invention, other and further embodiments of the invention may be devised without departing from the basic scope thereof, and the scope thereof is determined by the claims that follow.

What is claimed is:

1. A crystallization point test apparatus comprising:
a test vial for containing a fluid sample in an interior of the vial, wherein the test vial is light permeable;
a cooling source for cooling the fluid sample;
a temperature sensor positioned in the interior of the test vial for measuring the temperature of the fluid sample;
an external light source configured to direct light into the test vial; and
an external light detector configured to measure the amount of light that traverses both the test vial and a portion of the fluid sample.

2. The crystallization point test apparatus of claim 1 further comprising a stirrer positioned in the interior of the vial for circulating the fluid sample.

3. The crystallization point test apparatus of claim 1, wherein the cooling source is selected from the group consisting of a Peltier junction, a cooling jacket, a cooling bath, and a cooling magnetic stir-plate.

4. The crystallization point test apparatus of claim 1, wherein the external light source is selected from the group consisting of a laser, lamp, and LED.

5. The crystallization point test apparatus of claim 1, wherein the external light source and the external light detector are positioned outside the test vial and laterally spaced such that a light beam emanating from the external light source may travel in a single pass from the external light source to the external light detector.

6. The crystallization point test apparatus of claim 1, wherein the portion of the fluid sample that light traverses has a volume of at least about 2 ml.

7. The crystallization point test apparatus of claim 1, further comprising a computer for monitoring the cooling source, the temperature sensor, the external light source, and the external light detector.

8. A crystallization point test apparatus comprising:
a test vial for containing a fluid sample in an interior of the vial, wherein the test vial is light permeable;
a cooling source for cooling the fluid sample, wherein the cooling source is selected from the group consisting of a Peltier element and a cooling magnetic stir-plate;
a temperature sensor positioned in the interior of the test vial for measuring the temperature of the fluid sample;
an external light source configured to direct light into the test vial; and
an external light detector configured to measure the amount of light that traverses both the test vial and a portion of the fluid sample.

9. The crystallization point test apparatus of claim 8 further comprising a stirrer positioned in the interior of the test vial for circulating the fluid sample.

10. The crystallization point test apparatus of claim 8, wherein the external light source is selected from the group consisting of a laser, lamp, and LED.

11. The crystallization point test apparatus of claim 8, wherein the external light source and the external light detector are positioned outside the test vial and laterally spaced such that a light beam emanating from the light source may travel in a single pass through the portion of the fluid sample and into the light detector.

12. The crystallization point test apparatus of claim 8, wherein the portion of the fluid sample that light traverses has a volume in the range from about 4 ml to about 10 ml.

13. The crystallization point test apparatus of claim 8, further comprising a computer for monitoring the cooling source, the temperature sensor, the external light source, and the external light detector.

14. A method of determining a crystallization temperature of a fluid, comprising:
providing a test vial containing a fluid sample, wherein the test vial is light permeable;
positioning a temperature sensor into the fluid sample for measuring the temperature of the fluid sample;
directing a beam of light into the test vial, wherein the beam of light travels from a light source positioned outside the test vial and towards the fluid sample;
optically detecting the degree of crystallization in the fluid sample by measuring the amount of the beam of light that travels through a portion of the fluid sample and into a light detector, wherein the light detector is positioned outside the test vial; and
cooling the fluid sample.

15. The method of claim 14, wherein the cooling the fluid sample is achieved using a cooling source, and wherein the cooling source is selected from the group consisting of a Peltier element and a cooling magnetic stir-plate.

16. The method of claim 14, wherein the beam of light comprises at least one of visible light, polarized light, laser light, IR light and UV light.

17. The method of claim 14, wherein the portion of the fluid sample that light traverses has a volume of at least about 4 ml.

18. The method of claim 14, further comprising using a computer to automate the cooling of the fluid sample by monitoring with the computer the degree of crystallization optically detected by the light detector.

19. The method of claim 14, further comprising:
optically detecting the onset of crystal formation in the fluid sample while measuring the corresponding temperature;
stopping the cooling of the fluid sample immediately after the onset of crystal formation;
optically detecting the progression of crystal formation in the fluid sample while measuring the corresponding temperature;
heating the fluid sample; and
optically detecting the dissolution of crystals in the fluid sample while measuring the corresponding temperature.

20. The method of claim 19, wherein the measuring the corresponding temperature comprises recording at least one temperature in the group consisting of FCTA, TCT, and LCTD.

21. The method of claim 19, further comprising using a computer to automate the cooling and heating of the fluid sample by monitoring with the computer the temperature of the fluid sample measured by the temperature sensor and the degree of crystallization optically detected by the light detector.
